(19) United States
(12) Patent Application Publication
(10) Pub. No.: US 2023/0062794 A1
(43) Pub. Date: Mar. 2, 2023

(54) HEALTH MONITORING PLATFORM

(71) Applicant: Oura Health Oy, Oulu (FI)

(72) Inventors: Brian James Gilan, San Francisco, CA (US); Tero Teemu Eemeli Kurppa, Helsinki (FI); Ville Petten Saarinen, Helsinki (FI); Utkarsh Ramu Raut, Espoo (FI); Ossi Tapio Hanhinen, Espoo (FI); Ivan Uladzimiravich Markevich, Espoo (FI); Oyelowo Oyedayo, Vantaa (FI); Marianne Elina Siren, Kirkkonummi (FI); Janne Olavi Haapsaari, Vantaa (FI)

(21) Appl. No.: 17/892,954

(22) Filed: Aug. 22, 2022

Related U.S. Application Data
(60) Provisional application No. 63/238,888, filed on Aug. 31, 2021.

(51) Int. Cl. G16H 40/20; G16H 40/67; G16H 50/70; G16H 20/00

(52) U.S. Cl. CPC G16H 40/20; G16H 40/67; G16H 50/70; G16H 20/00

(57) ABSTRACT

Methods, systems, and devices for a health monitoring platform are described. A method may include receiving physiological data associated with one or more users, the physiological data being continuously collected via one or more wearable devices associated with the respective users. The method may include receiving, via one or more user devices associated with the one or more users, supplemental data associated with the physiological data for the one or more users, the supplemental data comprising indications of events, subjective attributes, or both. The method may include causing a graphical user interface (GUI) of an administrator device to display at least a portion of the physiological data and at least a portion of the supplemental data in conjunction with the physiological data, where the GUI indicates one or more respective subsets of the physiological data that correspond to one or more respective subsets of the supplemental data.

### CROSS REFERENCE

The present application for patent claims the benefit of U.S. Provisional Patent Application No. 63/238,888 by GILAN et al., entitled “HEALTH MONITORING PLATFORM,” filed Aug. 31, 2021, assigned to the assignee thereof, and expressly incorporated by reference herein.

### FIELD OF TECHNOLOGY

The following relates to wearable devices and data processing, including a health monitoring platform including wearable devices.

### BACKGROUND

Some wearable devices may be configured to collect physiological data from users, including temperature data, heart rate data, and the like. Many users have a desire for more insight regarding their physical health.

### BRIEF DESCRIPTION OF THE DRAWINGS

FIG. 1 illustrates an example of a system that supports a health monitoring platform in accordance with aspects of the present disclosure.

FIG. 2 illustrates an example of a system that supports a health monitoring platform in accordance with aspects of the present disclosure.

FIG. 3 illustrates an example of a health monitoring platform in accordance with aspects of the present disclosure.

FIG. 4 illustrates an example of a graphical user interface (GUI) that supports a health monitoring platform in accordance with aspects of the present disclosure.

FIG. 5 illustrates an example of a GUI that supports a health monitoring platform in accordance with aspects of the present disclosure.

FIG. 6 illustrates an example of a GUI that supports a health monitoring platform in accordance with aspects of the present disclosure.

FIG. 7 illustrates an example of a GUI that supports a health monitoring platform in accordance with aspects of the present disclosure.

FIG. 8 illustrates an example of a GUI that supports a health monitoring platform in accordance with aspects of the present disclosure.

FIG. 9 shows a block diagram of an apparatus that supports a health monitoring platform in accordance with aspects of the present disclosure.

FIG. 10 shows a block diagram of a wearable application that supports a health monitoring platform in accordance with aspects of the present disclosure.

FIG. 11 shows a diagram of a system including a device that supports a health monitoring platform in accordance with aspects of the present disclosure.

FIGS. 12 through 15 show flowcharts illustrating methods that support a health monitoring platform in accordance with aspects of the present disclosure.

### DETAILED DESCRIPTION

Some wearable devices may be configured to collect data from users for the purposes of health monitoring. For example, some wearable devices may be configured to continuously acquire physiological data associated with a user including temperature data, heart rate data, and the like. In order to efficiently and accurately track physiological data, a wearable device may be configured to collect data continuously while the user wears the device.

Most individuals do not have the capability to continually monitor their health. Moreover, most administrators (e.g., doctors, coaches, managers, employers) do not have the capability to continually monitor the health of each user within a group monitored by the administrator. For example, the administrators do not have the capability to view and/or analyze physiological data and metrics for each user across the group. Most individuals go to the doctor a few times a year, and of those individuals, few to none may provide their results to the administrator. As such, most individuals and administrators only have several snapshots of their health-related data (e.g., temperature, heart rate, blood pressure, etc.) or health-related data of the user at a few points in time throughout the year. The limited data points, along with the infrequent and inconsistent times that the data points are collected, provide a very limited view of the user's overall health. As such, conventional techniques for health monitoring are deficient for multiple reasons.

Accordingly, to facilitate improved health monitoring, aspects of the present disclosure are directed to a health monitoring platform configured to continuously collect physiological data from users to provide continuous health monitoring. In particular, aspects of the present disclosure are directed to techniques for causing a graphical user interface (GUI) of an administrator device to display at least a portion of supplemental data (e.g., data “tags”) in conjunction with the physiological data, and alerting users and/or administrators of a potential health risk when the physiological data satisfies one or more thresholds (e.g., temperature exceeding a temperature threshold, HRV dropping below a threshold, Sleep/Readiness Scores dropping below a threshold).

In some implementations, wearable devices may be used to continuously acquire physiological data and other data (e.g., supplemental data, or data “tags”) from a group of users for improved health monitoring. The wearable devices may be used to receive supplemental data associated with the physiological data for the one or more users of the group of users. The supplemental data may include an indication of events, subjective attributes, one or more tags associated with the physiological data, or the like. The respective users and/or an administrator of the group of users may have access to the acquired physiological data and/or supplemental data. In some implementations, users may be able to view their physiological data and/or supplemental data where the GUI indicates one or more respective subsets of the physiological data that correspond to one or more respective subsets of the supplemental data.

For example, an administrator associated with a group of users may access a user interface that displays the physiological data and/or supplemental data for each user of the group. In some examples, the administrator may know the identity of each user such that the administrator may easily monitor group metrics and spot anomalies between multiple users within the group. For instance, the administrator may be able to see cases where a given user input a “stress/anxiety” tag (e.g., supplemental data), and may be able to see how the user's sleep was affected on days that the user input the “stress/anxiety” tag. By sharing the user data with a small, well-defined group for specific purposes (e.g., a work cohort, an athletic sports team), the privacy and security for each of the respective users may be preserved.

In some implementations, an administrator of a group of users may set or define thresholds for each metric or score, where the thresholds may trigger messaging, alerts, or other actions when the respective thresholds are satisfied. For example, if the health monitoring system determines that a health risk metric for a user satisfies (e.g., exceeds) a threshold, thereby indicating a presence of a potential health risk, the system may automatically trigger transmission of a message to the user and/or an administrator. In some examples, the health monitoring platform may tell the administrator which user is associated with the potential health risk so that the administrator may directly contact the user.

Aspects of the disclosure are initially described in the context of systems supporting physiological data collection from users via wearable devices. Additional aspects of the disclosure are described in the context of example GUIs. Aspects of the disclosure are further illustrated by and described with reference to apparatus diagrams, system diagrams, and flowcharts that relate to health monitoring platform.

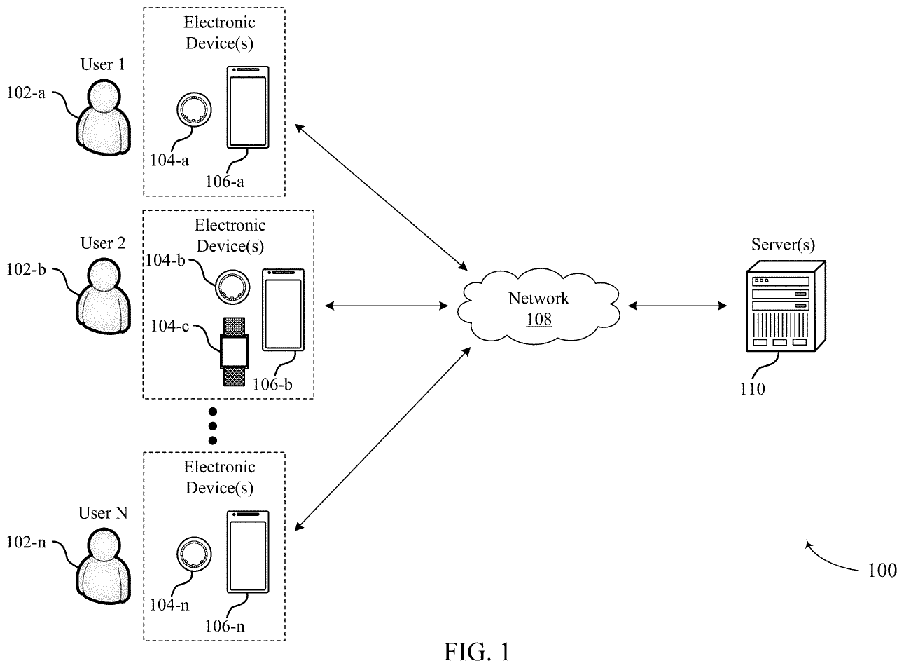

FIG. 1 illustrates an example of a system 100 that supports a health monitoring platform in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting diodes (LEDs) (e.g., red LEDs, green LEDs) that emit light on the palm-side of a user's finger to collect physiological data based on arterial blood flow within the user's finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for monitoring health metrics by using a wearable device. In particular, the system 100 illustrated in FIG. 1 may support techniques for analyzing metrics for groups of users 102, and notifying the user 102 if the physiological data satisfies a threshold. For example, as shown in FIG. 1, User 1 (e.g., user 102-a) may be associated with a wearable device (e.g., ring 104-a) and a user device 106-a. In this example, the ring 104-a may collect physiological data associated with the user, including temperature, heart rate, HRV, and the like. In some aspects, data collected by the ring 104-a may be used to determine whether the physiological data of User 1 satisfies a threshold. In some examples, the user device 106 may receive supplemental data associated with the physiological data for User 1. The supplemental data may include indications of events, subjective attributes, one or more tags associated with the physiological data (e.g., a “tag” indicating caffeine or alcohol consumption, food consumption, stress/anxiety, poor sleep, etc.), or a combination thereof.

Detection of physiological data and supplemental data and calculation of health metrics may be performed by any of the components of the system 100, including the ring 104-a, the user device 106-a associated with User 1, the one or more servers 110, or any combination thereof. The system 100 may be configured to receive physiological data and tags from users 102, and display the physiological data and tags in a GUI of an administrator device. The system 100 may also indicate which subsets of the physiological data correspond to which tags. For example, if an administrator clicks on a specific tag, the system 100 may show which days each user 102 experienced the specific tag. For instance, by clicking an “alcohol” tag, the administrator may be able to see physiological data collected on days on which users indicated that they consumed alcohol, which may enable the administrator to efficiently determine how alcohol affects the respective users (e.g., how alcohol affects sleep quality or duration).

In some cases, calculated health metrics may be compared to thresholds to perform the health monitoring techniques described herein. In some cases, the thresholds may be set or adjusted by an administrator based on a relative acceptable health metric for each user 102 in the system (e.g., User 1, User 2, . . . , User N). Upon detecting that the physiological data for a user 102 satisfies a threshold, the system 100 may notify the user 102 and/or a system administrator of a potential health risk or other information associated with the physiological data. For example, upon detecting that the health metric for a User 1 satisfies a threshold, the system 100 may display an alert of a potential health risk via the user device 106-a or a message associated with the physiological data. Moreover, the system 100 may monitor whether the User 1 viewed the notification of the potential health risk (e.g., via a GUI of the mobile device), and may selectively send a reminder accordingly (e.g., transmit a reminder or follow-up notification if the User 1 has not viewed the notification).

In some implementations, the alerts and messages associated with User 1 may be sent to User 1 (e.g., user device 106-a), an administrative personnel (e.g., manager of the group of users 102-a, 102-b, 102-c), or both. For example, the system 100 may generate alerts on the administrator user device 106-d when a user's data satisfies some threshold. For example, if a user's total sleep for a given timeframe drops into an unhealthy range, it may generate an alert for the administrator. In some cases, the administrator may input thresholds that may be used to trigger alerts. In some implementations, the system 100 may display messages to the user 102 when a user's data satisfies some threshold. For example, if a user's total sleep for a given timeframe drops into an unhealthy range, the system 100 may send a message to the user 102 that includes recommendations for improving sleep. In some implementations, the administrator may know the identity of User 1, and may transmit the notification to User 1 directly. In some cases, an administrator may input thresholds and pre-defined messages that may be delivered to users (e.g., “If a user's average deep sleep drops below this threshold, send the user this message”).

The messages may additionally, or alternatively, provide other insights regarding the potential health risk, such as health trends for User 1, contributing factors for the detected health risk, whether User 1 should consider contacting a medical professional, whether the user should stay home from work or limit other activities, whether their data is abnormal or reasons why it may be abnormal, links to documents/surveys, and medical guidance (e.g., here is a list of steps you can do to improve your overall health), and the like.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

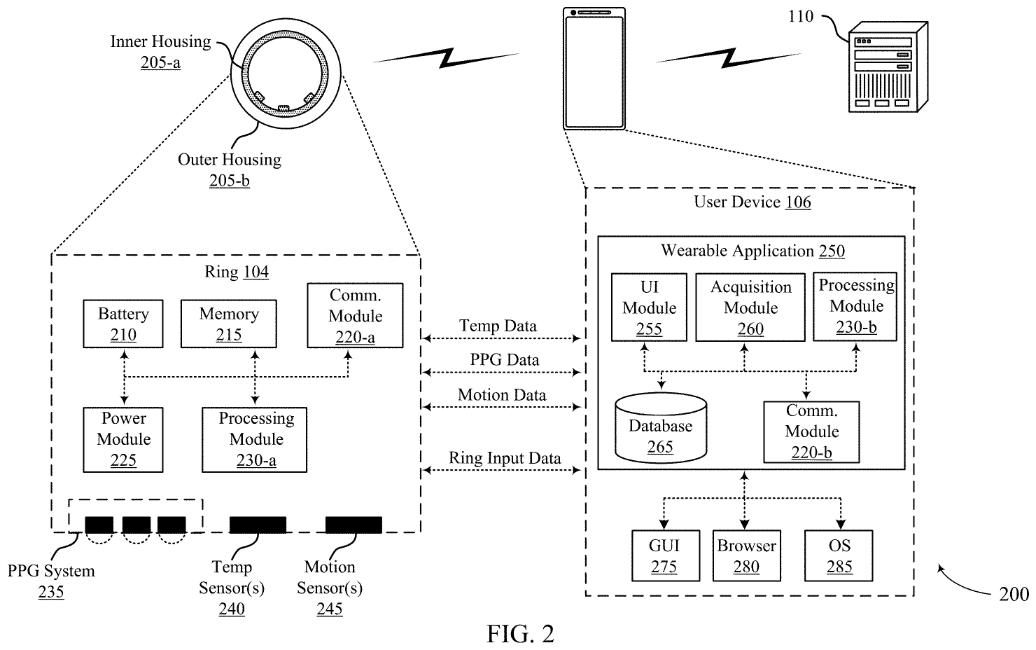

FIG. 2 illustrates an example of a system 200 that supports a health monitoring platform in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG LEDs. In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 that the optical receiver(s) receive transmitted light that is reflected through the region of the user's finger. In some implementations, the PPG system 235 may be configured as a transmissive PPG system 235 that the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner which is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for health monitoring. In particular, the respective components of the system 200 may be used to indicate which subsets of the physiological data correspond to which tags. For example, the system 200 may receive, via one or more user devices 106 associated with the user 102, supplemental data associated with physiological data for the user 102. The supplemental data may be an example of a tag (e.g., indication of event, subjective attributes, or both). For instance, a user 102 may be able to input tags that indicate alcohol consumption, headaches, stress/anxiety, cold/flu symptoms, etc. The system 200 may receive physiological data associated with one or more users 102. In some cases, the physiological data may be continuously collected via one or more wearable devices associated with the respective user 102.

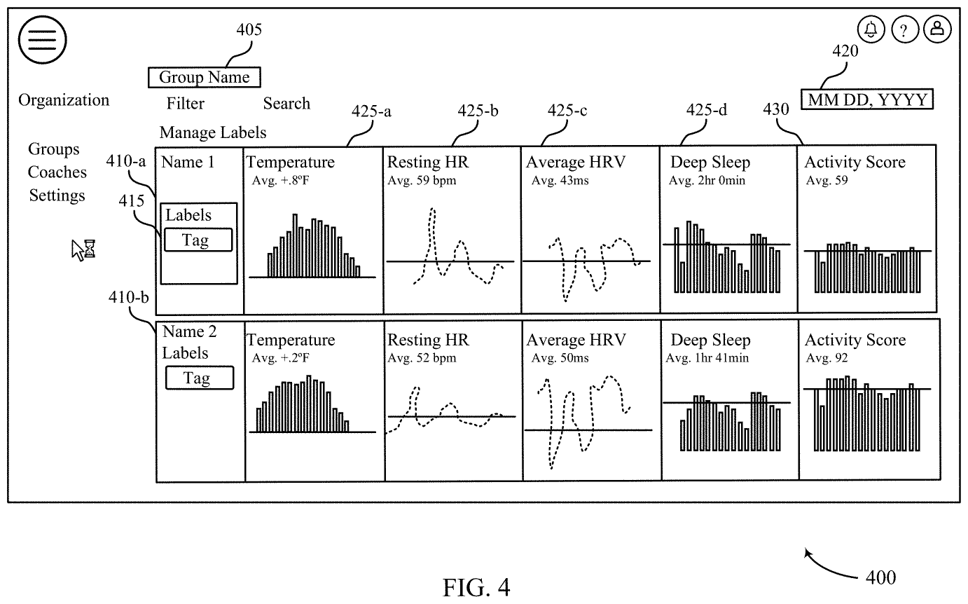

For example, as noted previously herein, the ring 104 of the system 200 may be worn by a user to collect data from the user, including temperature, heart rate, and the like. The ring of the system 200 may collect the physiological data from the user based on arterial blood flow. The physiological data may be collected continuously. In one example, the processing module 230-a may sample the user's temperature continuously throughout the day and night. Sampling at a sufficient rate (e.g., one sample per minute) throughout the day may provide sufficient temperature data for analysis described herein. In some implementations, the ring 104 may continuously acquire physiological data (e.g., at a sampling rate). Different physiological parameters may be collected/sampled at different sampling rates. For example, temperature may be sampled once per minute, whereas heart rate may be sampled more frequently, such as a rate of 250 Hz. Data collected by the ring 104 may be used to cause the GUI of an administrator device to display at least a portion of the supplemental data in conjunction with the physiological data. The GUI may indicate one or more respective subsets of the physiological data which correspond to one or more respective subsets of the supplemental data, that may be further shown and described with reference to FIG. 4.

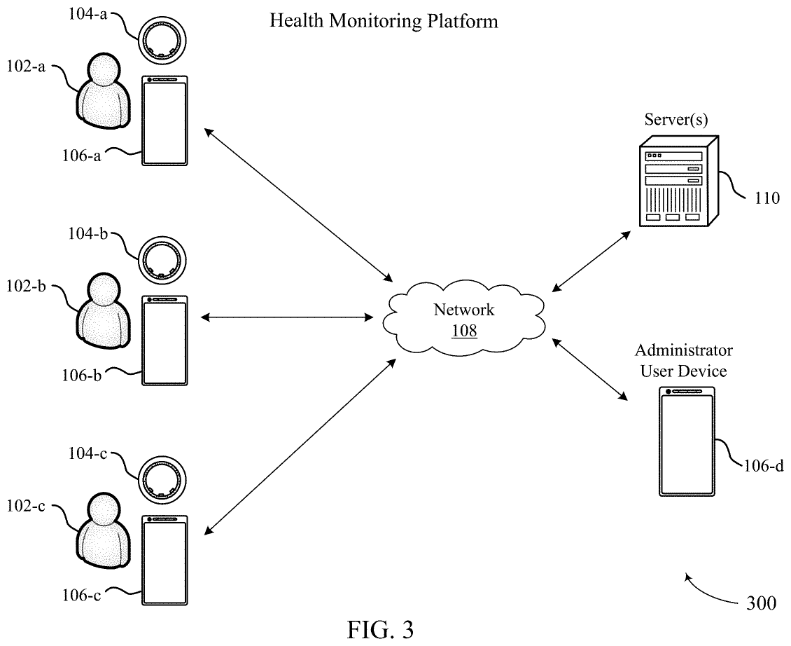

FIG. 3 illustrates an example of a health monitoring platform 300 in accordance with aspects of the present disclosure. The health monitoring platform 300 may implement, or be implemented by, aspects of the system 100, system 200, or both. In particular, health monitoring platform 300 illustrates an example of one or more rings 104 and user devices 106, as described with reference to FIGS. 1 and 2. In some examples, health monitoring platform 300 may represent a health monitoring platform for detecting illness or other medical conditions in one or more users 102.

In some examples, the health monitoring platform 300 may collect data from one or more users 102 via a wearable device 104, such as a ring. Each wearable device 104 may communicate with one or more user devices 106, such as a computer, cellular device, tablet, another wearable device 104, or other user device 106 of a user 102. For example, wearable device 104-a, that may include a ring or one or more other body-worn sensors, may acquire physiological data from the user 102-a and transmit the acquired physiological data to the user device 106-a and/or server 110 for processing. The user device 106-a may then display the processed physiological data and/or other determined parameters/metrics to the user 102-a. Similarly, wearable device 104-b may send data to user device 106-b for display to user 102-b. Wearable device 104-c may send data to user device 106-c for display to user 102-c. In some examples, the health monitoring platform 300 may include any quantity of users 102, such as User 1 through User N (e.g., with wearable device 104-n and user device 106-n). Each user 102 may have any number of wearable devices 104 collecting data, such as sleep data, heart rate data, HRV data, respiratory rate data, temperature data, blood oxygen data, etc.

The wearable devices 104, user devices 106, or both, may communicate with one or more servers 110 and/or an administrator user device 106-d via a network 108. For example, the wearable devices 104 may communicate directly with the servers 110 via network 108 by transmitting data collected from a user 102 directly to the servers 110 via the network 108. In some other examples, a wearable device 104 may connect with a user device 106 (e.g., via Bluetooth) to send the data collected from a user 102, where the user device 106 may forward the data to the servers 110 via the network 108. For example, wearable device 104-a may collect data from user 102-a (e.g., physiological data) and may send the data to the user device 106-a, where the user device 106-a may forward the data to the server 110 and/or administrator user device 106-d via network 108. The servers 110 may include databases or data stores (e.g., an application server, a database server, a cloud-based server, a datacenter, or a combination of these or other devices or systems of devices) configured to store received data and/or perform the various processing functions described herein. In this regard, the server 110 may be used for data storage, management, and processing.

In some examples, the components of the health monitoring platform 300 (e.g., user devices 106-a, 106-b, 106-c, servers 110, administrator user device 106-d) may be configured to acquire physiological data from each of the respective users 102, and may calculate scores or metrics associated with each respective user 102. Scores/metrics that may be calculated for each respective user 102 may include Sleep Scores, Readiness Scores, health risk metrics (e.g., illness risk scores, health risk metrics), and the like. In some aspects, scores/metrics may be calculated once a day (e.g., once in a 24 hour period), such as when a user 102 accesses the data for the first time each day or at a default time each day. Additionally, or alternatively, the health monitoring platform 300 may be configured to transmit determined scores/metrics to the administrator user device 106-d at regular or irregular periodicity (e.g., once a day, once per hour). In some implementations, the system may calculate different health risk scores for different health conditions or illnesses (e.g., a first health risk score associated with the flu, a second health risk score associated with a heart condition). Additional details associated with the calculation of metrics/scores for each user 102 are described in further detail with respect to FIG. 4.

In some implementations, physiological data and determined scores/metrics (e.g., health risk metrics) for each respective user may be presented to an administrator (e.g., doctor, health care professional, coach, manager, employer) via the administrator user device 106-d in a non-anonymized manner or an anonymized manner. In the context of non-anonymized health monitoring, health information (e.g., physiological data, health risk scores) may be presented via the administrator user device 106-d along with identifiers (e.g., non-anonymized user identifiers) for each respective user. In other words, in the context of non-anonymized health monitoring, the administrator may be able to know which physiological data and health risk metrics correspond to each respective user 102. Comparatively, in the context of anonymized health monitoring, health information (e.g., physiological data, health risk scores) may be presented via the administrator user device 106-d along with anonymized user identifiers for each respective user 102. In other words, in the context of anonymized health monitoring, the administrator may not know which physiological data and health risk metrics correspond to each respective user 102. Anonymized health monitoring techniques may facilitate improved user privacy, and may protect user health information.

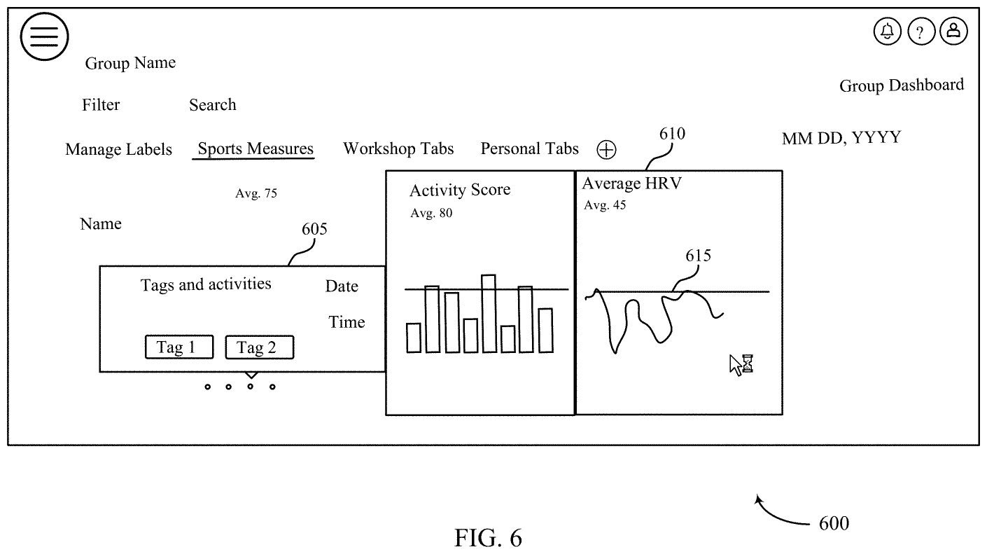

In some examples, an administrative personnel operating the administrator user device 106-d (e.g., a user of user device 106-d) may set/adjust a threshold for each category of collected data, for one or more scores calculated from the collected data, or both. For example, an administrator may be able to define (e.g., via the administrator user device 106-d) one or more pre-defined thresholds for evaluating health risk metrics determined for the respective users 102. In some other examples, the health monitoring platform 300 (e.g., server 110, administrator user device 106-d) may select/determine the one or more pre-defined thresholds by default, and may communicate the pre-defined thresholds to the administrator user device 106-d. Additionally, or alternatively, the pre-defined thresholds may be defined (e.g., preconfigured) at the administrator user device 106-d. In some examples, the pre-defined thresholds may be based on one or more uncertainty rates (e.g., false-positive rates, false-negative rates, true-positive rates, true-negative rates) related to illness detection and health monitoring for the users 102 of the health monitoring platform 300. The selection of pre-defined thresholds will be described in further detail with respect to FIG. 6.

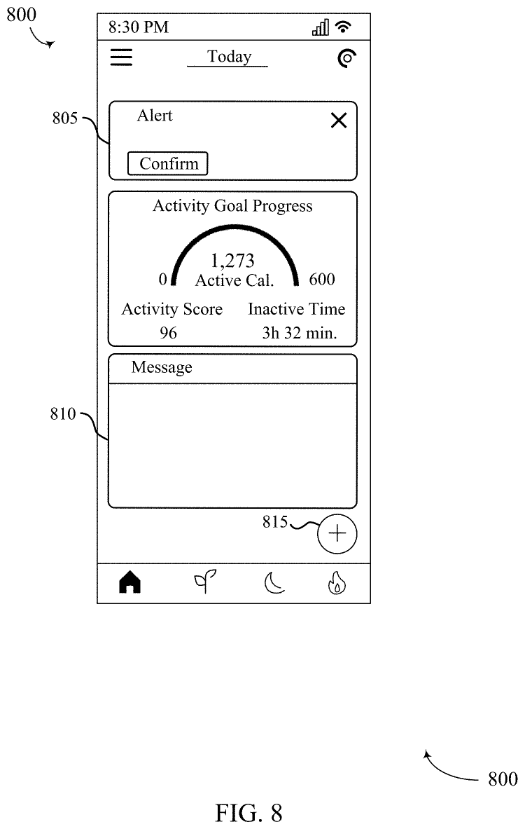

In cases where collected physiological data and/or determined scores/metrics (e.g., health risk metrics) for a user 102 of the health monitoring platform 300 satisfy a pre-defined threshold, and thereby indicate a potential health risk, the health monitoring platform 300 (e.g., server 110, administrator user device 106-d) may transmit a message or notification to the user 102 associated with the potential health risk. For example, if user 102-b exhibits a health risk metric above a pre-defined threshold, the server 110 may send a message to user device 106-b (e.g., via electronic communication, a push notification to an application running at user device 106-b, or the like). Notifications of potential health risks that may be transmitted to the respective user devices 106-a, 106-b, 106-c as will be further shown and described with respect to FIG. 8. In some cases, messages transmitted to the user devices 106-a, 106-b, 106-c may be customizable (e.g., via the administrator user device 106-d), which will be described in further detail with respect to FIG. 8. Moreover, an administrator associated with the administrator user device 106-d may track whether respective users 102 have viewed messages related to potential health risks, which will be described in further detail with respect to FIG. 8.

In some cases, the health monitoring platform 300 (e.g., server 110, administrator user device 106-d) may receive physiological data associated with the user 102. The health monitoring platform 300 may receive, via a user device 106 associated with the user 102, supplemental data (e.g., events, subjective attributes, tags, and the like) associated with the physiological data. In some cases, the health monitoring platform 300 may cause a GUI of an administrator user device 106-d to display at least a portion of the physiological data and at least a portion of the supplemental data in conjunction with the physiological data. The GUI may indicate respective subsets of the physiological data which correspond to respective subsets of the supplemental data, which will be described in further detail with respect to FIG. 4.

FIG. 4 illustrates an example of a GUI 400 that supports a health monitoring platform in accordance with aspects of the present disclosure. The GUI 400 may implement, or be implemented by, aspects of the system 100, system 200, health monitoring platform 300, or any combination thereof. For example, the GUI 400 may be an example of a GUI of the administrator user device 106-d of the health monitoring platform 300 illustrated in FIG. 3.

The GUI 400 illustrates an application page that may be displayed to an administrator/user via the GUI 400 (e.g., GUI of the administrator user device 106-d). Continuing with the example above, an administrator associated with a group 405 of users may check collected data or calculated scores for one or more users in the group 405. The group 405 may be based on a physical location of one or more users (e.g., a floor in an office building), one or more lifestyle parameters for each user 102 (e.g., users with known health risks), employer or organization, athletic team, or the like. The administrative personnel may be presented with the application page upon opening a platform (e.g., “app,” wearable application 250) for viewing collected data from wearable devices 104 (e.g., rings), calculated scores based on the collected data, or both, for the group 405.

In some examples, the administrative personnel may view a user profile 410 for each user 102 in the group 405 (e.g., a profile that identifies a user identity or an anonymized user identifier for each respective user). If the user profile 410 includes identity information of the user, the administrative personnel may have access to health data of the users (e.g., non-anonymized health monitoring). In other words, in the context of non-anonymized health monitoring, Sleep/Readiness/Activity Scores 430 and/or acquired physiological data 425 may be presented to an administrator along with non-anonymized user identifiers such that the administrator may determine which users correspond to the respective metrics/scores, acquired data, and the like. However, if the administrative personnel is an employer, sharing health data may compromise privacy and ethical policies (e.g., by asking a user 102 to share potentially personal information). As such, in some cases, the user profile 410 may be anonymized such that the administrator may view the physiological data 425 and respective scores 430, but does not know the actual identity of the user associated with the user profile 410.

In such cases, the health monitoring platform 300 may enable the administrator or user 102 to view the physiological data 425 for the respective calendar day or calendar range upon selecting the user profile 410. For example, the health monitoring platform 300 may update the GUI 400 from displaying a group-level view (e.g., including user profile 410-a and user profile 410-b) to displaying an individual view (e.g., including user profile 410-a or user profile 410-b). In some cases, the health monitoring platform 300 may be able to search for a user 102 of the group 405. In other examples, the health monitoring platform 300 may be able to search for a user 102 across multiple groups 405.

In some examples, one or more users may opt-in to the group 405 by using a wearable device that collects health related data (e.g., physiological data or biometrics). In other cases, users may opt-into the group 405 for health tracking by inputting a user input (e.g., command) into a user device 106 associated with the respective user. For example, the users 102 of the health monitoring platform 300 may be able to opt-in to the health monitoring platform 300 (e.g., opt-in to the group 405) for health tracking by inputting one or more opt-in commands via the user devices 106 associated with each of the respective users. In some implementations, physiological data will not be collected (and scores/metrics will not be calculated) for a given user 102 until the user 102 has opted-into the health monitoring platform 300.

An administrative personnel (e.g., employer administrators) may access a dashboard, illustrated by GUI 400, that illustrates a list of user profiles 410 including at least user profile 410-a and user profile 410-b. In some cases, the administrative personnel may select the user profile 410 associated with a respective user 102 in order to view more details regarding the user's physiological data 425 and/or determined scores 430. For example, by selecting the user profile 410, an administrator may be able to view the user's physiological data 425 and activity score 430 for some historical time period (e.g., the last three weeks) as indicated by calendar range 420.

In some examples, the physiological data 425 may be collected continuously from wearable devices 104. Techniques described herein may continuously collect the physiological data from the user 102 based on arterial blood flow. In some implementations, the computing devices may sample the user's physiological data continuously throughout the day and night. Sampling at a sufficient rate for each respective physiological parameter (e.g., sampling temperature once per minute, sampling other physiological parameters at a higher sampling rate) throughout the day may provide sufficient data for analysis described herein. In some implementations, continuous data measurement at the finger may capture minute-to-minute or hour-to-hour temperature fluctuations that provide additional insight that may not be provided by other measurements elsewhere in or on the body.

As shown in FIG. 4, the GUI 400 may display physiological data 425 for each user. The physiological data 425 may be an example of temperature deviation (e.g., physiological data 425-a), lowest resting heart rate (e.g., physiological data 425-b), average HRV (e.g., physiological data 425-c), and deep sleep time (e.g., physiological data 425-d). The GUI 400 may display an average value, a baseline value, a graphical display (e.g., histogram, line graph, etc.), and the like associated with the physiological data 425 for the users of the group 405.

The health monitoring platform 300 may receive physiological data 425 and supplemental data 415 (e.g., labels, tags, etc.) from users and display the physiological data 425 and supplemental data 415 in the GUI 400 of an administrator user device 106-d. For example, the health monitoring platform 300 may receive the supplemental data 415 associated with the physiological data 425 for the one or more users where the supplemental data 415 includes indications of events, subjective attributes, or both.

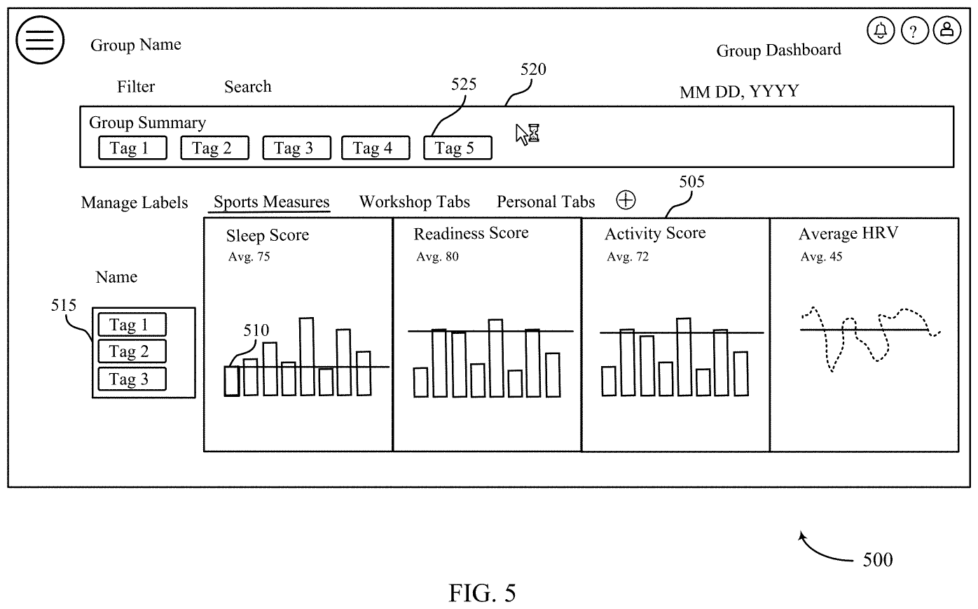

The health monitoring platform 300 may cause the GUI 400 of the administrator device to display at least a portion of the physiological data 425 and cause the GUI 400 of the administrator device to display at least a portion of the supplemental data 415 in conjunction with the physiological data 425. In such cases, the administrator user device 106-d may indicate which subsets of the physiological data 425 correspond to which subsets of supplemental data 415. For example, if an administrator selects a specific tag (e.g., supplemental data 415), then the GUI 400 may display which days the physiological data 425 may be associated with the specific tag, which is further illustrated with respect to FIG. 5.

Illustrating the user's historical physiological data 425 and activity scores 430 may enable the administrator to gauge the overall health of the user. In particular, historical physiological data 425 and activity scores 430 may enable the administrator to determine whether a physiological data 425 metric or parameter for the user 102 is a random one-off value, or if the user 102 has exhibited a trend associated with the physiological data 425. In some implementations, additional health data that is displayed upon selection of the user profile 410 may be presented.

In some cases, the health monitoring platform 300 may track trends within the group 405 (e.g., trends between different users of the group 405) and download physiological data 425 in different file formats (e.g., Microsoft Word documents, Microsoft Excel Spreadsheets, PDFs, etc.). The health monitoring platform 300 may be able to assemble the group 405 and view data for the group 405. In some cases, an organization may include more than one group 405 (e.g., a group for the entire organization, groups for different floors, groups for different teams within the organization that frequently interact with one another, etc.).

The health monitoring platform 300 may automatically generate a health risk metric for at least a subset of users in the group 405 when the wearable device syncs collected data. Health risk metrics may be calculated for each respective user of the group 405 based on physiological data collected from each respective user. For example, the GUI 400 may display one or more health risk metrics for each user. As used herein, the terms “health risk metric,” and like terms, may refer to scores or other metrics that indicate a relative probability or likelihood that a given user 102 may experience some health risk (e.g., probability that a user 102 is or will become ill, relative probability that the user 102 will experience a cardiac episode, relative probability that a user is at risk of sleep apnea, relative probability that a user 102 may develop type two diabetes).

In some examples, upon identifying that a user 102 may exhibit a potential health risk, the health monitoring platform 300 may transmit a configurable message and/or alert to the user device 106 corresponding to the respective user, which is further illustrated with respect to FIG. 8.

FIG. 5 illustrates an example of a GUI 500 that supports a health monitoring platform in accordance with aspects of the present disclosure. The GUI 500 may implement, or be implemented by, aspects of the system 100, system 200, health monitoring platform 300, or any combination thereof. For example, the GUI 500 may be an example of a GUI of the administrator user device 106-d of the health monitoring platform 300 illustrated in FIG. 3.

The GUI 500 illustrates an application page that may be displayed to an administrator/user via the GUI 500 (e.g., GUI of the administrator user device 106-d). Continuing with the example above, the health monitoring platform 300 may receive supplemental data 515 (e.g., labels, tags, etc.) from users and display the physiological data 505 and supplemental data 515 in the GUI 500 of an administrator user device 106-d. For example, the health monitoring platform 300 may receive the supplemental data 515 associated with the physiological data 505 for the one or more users where the supplemental data 515 includes indications of events, subjective attributes, or both.

The health monitoring platform 300 may cause the GUI 500 of the administrator user device 106-d to display at least a portion of the physiological data 505 and cause the GUI 500 of the administrator user device 106-d to display at least a portion of the supplemental data 515 in conjunction with the physiological data 505 via indication 510. In such cases, the GUI 500 may highlight, via indication 510, one or more respective subsets of the physiological data 505 that correspond to one or more respective subsets of the supplemental data 515.

For example, the administrator user device 106-d may indicate which subsets of the physiological data 505 corresponds to which tags (e.g., supplemental data 515) for a user. In some cases, the administrator user device 106-d may indicate which subsets of the physiological data 505 corresponds to which tags (e.g., supplemental data 515) for multiple users within a group. In one example, if the health monitoring platform 300 receives a selection for an alcohol tag (e.g., supplemental data 515), the GUI 500 may indicate which days the user or users consumed alcohol (e.g., input an “alcohol” tag) by highlighting the corresponding days in the physiological data 505 via the indication 510.

The health monitoring platform 300 may receive, via a user device 106 of the user, an indication of the supplemental data 515. In other examples, the health monitoring platform 300 may automatically detect the supplemental data 515 based on the received physiological data 505. For example, an increased heart rate associated with the physiological data 505 may indicate that the user 102 is exercising such that the health monitoring platform 300 may receive an indication of exercise as the supplemental data 515.

The health monitoring platform 300 may determine what days users tag specific supplemental data 515 and what effect the supplemental data 515 has on the physiological data 505 and/or scores (e.g., Sleep Scores, Readiness Scores, Activity Scores) for the respective users. In such cases, the health monitoring platform 300 may determine a correlation between an activity (e.g., supplemental data 515) and the physiological data 505 and cause the GUI 500 to visually indicate the correlation. For example, the GUI 500 may provide insight to the user and/or administrator such that the indication 510 may highlight that alcohol was tagged as supplemental data 515 and the Sleep Score (e.g., physiological data 505) was lower for the calendar day that alcohol was tagged.

In some examples, the health monitoring platform 300 may receive, via an administrator user device 106-d, an indication of supplemental data 515. In other words, an administrator may be able to input supplemental data 515 (e.g., tags) for users within the group. For example, the health monitoring platform 300 may receive a tag such as difficult practice, travel day, or game day, and the health monitoring platform 300 may cause the GUI 500 to highlight, via indication 510, the corresponding instances of those tags in the physiological data 505 such that the user 102 and/or administrator may easily view the effects of the supplemental data 515 on the physiological data 505. For example, by inputting a “game day” tag via the administrator user device 102-d, the health monitoring platform 300 may enable the administrator to quickly view how a group of athletes Sleep Scores and other physiological data is affected the night before a game.

The GUI 500 may include a group summary 520 of tags and activities for a specified period of time. For example, the group summary 520 may include one or more supplemental data summaries 525 for the group. The group summary 520 may indicate a quantity of supplemental data summaries 525 for the group where each supplemental data summary 525 may indicate a quantity of instances that the health monitoring platform 300 received supplemental data 515. In some implementations, the health monitoring platform 300 may rank the supplemental data summaries 525 such that the GUI 500 displays the supplemental data summaries 525 in order of most frequently tagged supplemental data 515 to least frequently tagged supplemental data 515. In such cases, the health monitoring platform 300 may determine common activities for the group of users over a certain time period.

In some cases, the GUI 500 may display trends for a certain time period. For example, the health monitoring platform 300 may receive an indication to display trends for 1, 7, 14, or 28 days. The health monitoring platform 300 may enable administrators to monitor progress for multiple members over time and chart metrics most relevant to the group.

FIG. 6 illustrates an example of a GUI 600 that supports a health monitoring platform in accordance with aspects of the present disclosure. The GUI 600 may implement, or be implemented by, aspects of the system 100, system 200, health monitoring platform 300, or any combination thereof. For example, the GUI 600 may be an example of a GUI of the administrator user device 106-d of the health monitoring platform 300 illustrated in FIG. 3.

The GUI 600 illustrates an application page that may be displayed to an administrator/user via the GUI 600 (e.g., GUI of the administrator user device 106-d). Continuing with the example above, the health monitoring platform 300 may receive physiological data 610 and supplemental data 605 (e.g., labels, tags, etc.) from users and display the physiological data 610 and supplemental data 605 in the GUI 600 of an administrator user device 106-d. For example, the health monitoring platform 300 may receive the supplemental data 605 associated with the physiological data 610 for the one or more users where the supplemental data 605 includes indications of events, subjective attributes, or both.

The health monitoring platform 300 may cause the GUI 600 of the administrator device to display at least a portion of the physiological data 610 and cause the GUI 600 of the administrator device to display at least a portion of the supplemental data 605 in conjunction with the physiological data 610. The supplemental data 605 may include additional information and details associated with the physiological data 610. For example, the supplemental data 605 may include a time stamp, a date, and details associated with the supplemental data 605 (e.g., a type of activity, food or drink consumed the day the supplemental data 605 was received, etc.)

In some cases, the supplemental data 605 may be presented to administrators in a manner (e.g., including the details associated with the supplemental data 605 and/or the physiological data 610 including the threshold 615) that enables the administrator to have a complete view of the mental, physical, or emotional state of the user 102 of the group and visualize how the day-to-day behaviors affect the biometrics (e.g., physiological data 610). By aggregating passive data (e.g., physiological data 610) from multiple users within a group and incorporating subjective input (e.g., supplemental data 605) into the aggregated data, the overall health of each respective user 102 may be more efficiently monitored and improved.

In some implementations, the health monitoring platform 300 may determine that the physiological data 610 associated with a user 102 satisfies one or more thresholds 615. In such cases, the health monitoring platform 300 may cause the GUI 600 of the administrator user device 106-d to display an alert associated with the user 102 upon determining that the physiological data 610 associated with the user 102 satisfies the one or more thresholds 615. The health monitoring platform 300 may cause the GUI 600 of the user device associated with the user 102 to display a message based on determining that the physiological data 610 associated with the user 102 satisfies the one or more thresholds 615.

In some examples, administrative personnel may receive a notification (e.g., via electronic communication) if a user 102 in the group exhibits physiological data 610 that satisfies one or more thresholds 615 (e.g., pre-defined thresholds). The health monitoring platform 300 may contact users that exhibit physiological data 610 that satisfies threshold 615 by sending a message to the users. Additionally, or alternatively, the health monitoring platform 300 may transmit a notification to the administrator user device 106-d if a user 102 exhibits physiological data 610 that satisfies a threshold 615, as will be further discussed in FIG. 8.

For example, as shown in GUI 600, the health monitoring platform 300 may determine that a user 102 exhibits physiological data 610 (e.g., average HRV) that exceeds the threshold 615 (e.g., threshold HRV). In this example, the health monitoring platform 300 may transmit a signal/message to cause the administrator user device 106-d to display a notification of a potential health risk for the identified user. In other examples, the health monitoring platform 300 may determine that more than one user 102 exhibits physiological data 610 that exceeds the threshold 615. In this example, the health monitoring platform 300 may transmit a signal/message to cause the administrator user device 106-d to display a notification of a potential health risk for the more than one identified user, which is further illustrated with respect to FIG. 8.

Users with physiological data 610 above the threshold 615 may be referred to as “at-risk users” for a potential health risk, while users with physiological data 610 below the threshold 615 may not be at risk. In some cases, the administrative personnel may set and adjust the threshold 615. In some other cases, the threshold 615 may be set to a default physiological data 610, that may be referred to as a baseline threshold. The baseline threshold may be calculated from a 90-day rolling average. For example, according to the company-defined protocol, administrators may contact users with physiological data 610 above a customer-specific threshold 615 and may inquire about the situation of the user. If the administrator decides there may be a risk associated with the user, the user 102 may be sent to receive help from a medical professional. The administrative personnel may ask the user 102 to stay away from the rest of the group of users or rest until the health risk is resolved.

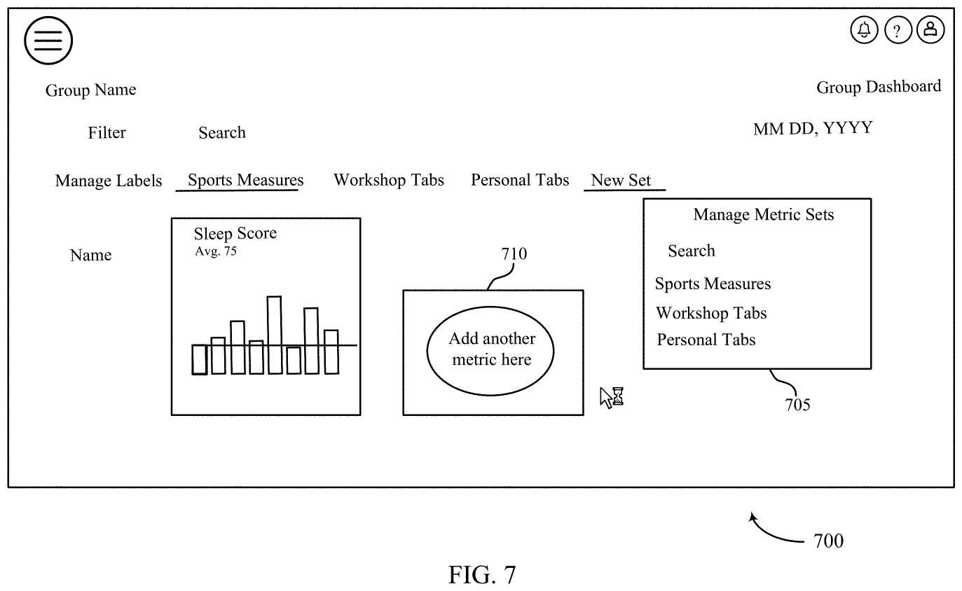

FIG. 7 illustrates an example of a GUI 700 that supports a health monitoring platform in accordance with aspects of the present disclosure. The GUI 700 may implement, or be implemented by, aspects of the system 100, system 200, health monitoring platform 300, or any combination thereof. For example, the GUI 700 may be an example of a GUI of the administrator user device 106-d of the health monitoring platform 300 illustrated in FIG. 3.

The GUI 700 illustrates an application page that may be displayed to an administrator/user via the GUI 700 (e.g., GUI of the administrator user device 106-d). Continuing with the example above, an administrator associated with a group of users may remove or add metrics of interest (e.g., data sets 705) from GUI 700. The administrator may define different data sets 705 that display different parameters of physiological data. For example, health monitoring platform 300 may receive, via the administrator user device 106-d, an indication of one or more data sets 705. Each data set 705 may include a set of parameters associated with the physiological data.

The health monitoring platform 300 may cause the GUI 700 of the administrator user device 106-d to display at least the portion of the physiological data corresponding to a data set 705. In one example, the data set 705 may include physiological data associated with a sports measure (e.g., Activity Score, Sleep Score, HRV, heart rate, and the like). Each respective data set 705 may include different sets of scores or physiological data parameters that are of interest to the administrator. For example, an administrator may define a “sleep data set” that enables the administrator to quickly view sleep-related scores and parameters for the users (e.g., Sleep Score, total sleep, sleep quality), as well as a “sports measures data set” that enables the administrator to quickly view performance-related scores and parameters for the users (e.g., Readiness Scores, Activity Scores, average respiratory rate, average HRV, etc.). The administrator may customize the view of the GUI 700 based on what view is relevant. For example, a coach (e.g., administrator) may select a “sports measure” metric to be displayed in the GUI 700 when putting together a training schedule for the upcoming week in order to more effectively tailor a team's training schedule based on the overall health and readiness of the team.

The health monitoring platform 300 may customize the GUI 700 to display any quantity of different data sets 705. For example, the administrator may select input 710 and, the health monitoring platform 300 may add another data set 705 for the GUI 700 to display. In such cases, the health monitoring platform 300 may configure a list of data sets 705 such that the user 102 (e.g., administrator) may toggle between different data sets 705.

FIG. 8 illustrates an example of a GUI 800 that supports a health monitoring platform in accordance with aspects of the present disclosure. The GUI 800 may implement, or be implemented by, aspects of the system 100, system 200, health monitoring platform 300, GUI 400, GUI 500, GUI 600, GUI 700, or any combination thereof. For example, the GUI 800 may be an example of a GUI of a user device 106 (e.g., user device 106-a, 106-b, 106-c) corresponding to a user 102 of the health monitoring platform 300 or an administrator user device 106-d illustrated in FIG. 3.

In some examples, the GUI 800 illustrates a series of application pages that may be displayed to a user 102 or administrator via the GUI 800 (e.g., GUI of the device 106-a, 106-b, 106-c, 106-d). As noted previously herein, physiological data collected from a user 102 may be used to calculate scores/metrics (e.g., health risk scores, Sleep Scores, Readiness Scores) for the respective user. Calculated scores/metrics may be displayed to the user 102 via a user device 106 corresponding to the user 102 or to the administrator via the administrator user device 106-d.

The server 110 of system may cause the GUI 800 of the user device 106 to display an alert 805 or message 810 (e.g., insights). In some implementations, the user device 106 and/or servers 110 may generate alerts 805 that may be displayed to the user 102 via the GUI 800. In some implementations, the user device 106 and/or servers 110 may generate messages 810 that may be displayed to the user 102 via the GUI 800. In some cases, the health monitoring platform 300 may indicate to the administrator (e.g., via the administrator user device 106-d) whether the user confirmed, viewed, and or dismissed the alert 805. In some cases, the administrator may be able to trigger duplicative or supplemental alerts.

Continuing with the example above, the user 102 may be presented with the application page upon opening the wearable application. As shown in FIG. 8, the administrator may input thresholds, via input 815, that may be used to trigger alerts 805. For example, the health monitoring platform 300 may receive, via the input 815 of the administrator user device 106-d, a user input including the one or more thresholds. In some cases, determining the satisfaction of the one or more thresholds may be based on receiving the user input.

In some cases, the health monitoring platform 300 may generate an alert 805 on the administrator user device 106-d when a user's data satisfies some threshold. For example, if a user's total sleep time for a given timeframe drops into an unhealthy range (e.g., below the threshold), the health monitoring platform 300 may generate an alert 805 for the administrator. In such cases, the health monitoring platform 300 may determine that the physiological data associated with a user 102 satisfies one or more thresholds and cause the GUI 800 of the administrator device to display an alert 805 associated with the user 102 based on determining that the physiological data associated with the user 102 satisfies the one or more thresholds.

The health monitoring platform 300 may display message 810 to the user 102 if a user's data satisfies some threshold. For example, if a user's total sleep time for a given timeframe drops into an unhealthy range (e.g., below the threshold), the health monitoring platform 300 may send a message 810 to the user. In such cases, the health monitoring platform 300 may cause the GUI 800 of a user device 106 associated with the user 102 to display a message 810 based on determining that the physiological data associated with the user 102 satisfies the one or more thresholds.

The administrator may input thresholds and pre-defined messages that may be delivered to users. For example, if a user's average deep sleep drops below the threshold, the health monitoring platform 300 may be configured to send the user 102 the message 810. The health monitoring platform 300 may receive, via the input 815 of the administrator device, a user 102 input including the one or more thresholds. The health monitoring platform 300 may receive, via the input 815 of the administrator device, one or more messages 810 associated with the one or more thresholds. The messages 810 may be configured to be communicated upon satisfaction of the respective one or more thresholds, the message included within the one or more messages 810. The GUI 800 of the user device 106 may display the message 810 based on receiving the user input and the one or more messages 810 via the administrator user device 106-d.

The messages 810 may include additional information associated with physiological data associated with the user. For example, the message 810 may include a link to an external document, a survey, medical guidance, or any combination thereof. The message 810 may inform the user 102 that their physiological data is normal or abnormal and, if abnormal, may provide health guidance as to a list of steps that the user 102 may do to improve their overall health.

In some examples, the alert 805 may be able to notify the user 102 if a metric (e.g., Sleep, Readiness, or Activity Score) is out of range. In such cases, the alert 805 and/or message 810 may serve as an accountability driver such that when the user 102 opens the application platform, the GUI 800 may display an alert 805 such as “Hey! We notice your Sleep Score is lower than average.” In such case, the message 810 may provide a recommendation such as “Here is a link to an article for some tips and tricks to getting a better night's sleep.”

For example, the health monitoring platform 300 (e.g., server 110, administrator user device 106-d) may transmit a message 810 to the user 102, where the message 810 is associated with a potential health risk for the user 102. For example, the user 102 may receive message 810, that may indicate for the user 102 to check in with a contact person associated with a group of users (e.g., administrator of an office). In some other cases, the user 102 may receive message 810, that may prompt the user 102 to check in for more information or dismiss the message 810. The messages 810 may be configurable/customizable, such that the user 102 may receive different messages 810 based on different health risk scores. Moreover, an administrator (e.g., administrator associated with the administrator user device 106-d) may customize the messages 810 that are to be transmitted to users 102 within the health monitoring platform 300.

In some implementations, the health monitoring platform 300 may be configured to receive physiological data and/or user inputs in order to train classifiers (e.g., supervised learning for a machine learning classifier) and improve messaging techniques. For example, the health monitoring platform 300 may assign a logical trigger that triggers when a message 810 is sent to the user device 106 and what is the content of the message 810. For example, if the Sleep Score is below a threshold for a certain amount of days, the health monitoring platform 300 may automatically generate the message 810 and transmit the message 810 to the user device 106.

By enabling administrators to define rules for sending messages 810 to users based on their respective health risk metrics, techniques described herein may automate alerts 805, messages 810, and other information that may be provided to users without divulging sensitive information to employer personnel or other administrators.

In some cases, administrative personnel may be able to see whether the user 102 has viewed or otherwise interacted with (e.g., opened, responded to) the alert 805 and/or message 810. In some cases, the user 102 may acknowledge the alert 805 and/or message 810. If the user 102 does not acknowledge or view the alert 805 and/or message 810, the administrative personnel may resend guidance (e.g., resend the alert 805 and/or message 810, or transmit a new alert 805 and/or message 810) or may mark the alert 805 and/or message 810 as viewed. In other words, the administrator may be able to trigger an additional alert 805 and/or message 810 that will be sent to the user device 106 of the respective user.

In some implementations, the health monitoring platform 300 may indicate one or more contributing factors that contribute to a user's health risk metric to provide additional insight regarding the user's health risk metric. For example, the application pages may indicate one or more physiological parameters (e.g., contributing factors) that resulted in the user's health risk metric, such as increased temperature, increased respiratory rate, increased heart rate, and the like. In other words, the health monitoring platform 300 may be configured to provide some information or other insights regarding determined health risk metrics. Personalized insights may indicate aspects of collected physiological data (e.g., contributing factors within the physiological data) that were used to generate the health risk metrics. In some cases, providing personalized insights may drive greater user engagement.

In some implementations, the health monitoring platform 300 may be configured to receive inputs 815 in order to train classifiers (e.g., supervised learning for a machine learning classifier) and improve illness detection techniques. For example, the user device 106 may display a health risk metric indicating a relative likelihood that the user 102 will become ill. Subsequently, the user 102 may input one or more user inputs, such as an onset of symptoms, a positive illness test, and the like. These user inputs may then be input into the classifier to train the classifier. In other words, the user inputs may be used to validate, or confirm, the determined illness risk metrics.

In some implementations, the health monitoring platform 300 may receive, via input 815 of the administrator user device 106-d, a restriction period that users may be unable to view their data. For example, the health monitoring platform 300 may receive an indication of a restriction period from the administrator user device 106-d. The restriction period may include a time duration that one or more users are unable to view physiological data collected via the wearable device associated with each respective user. The restriction period may be scheduled for a time when the health monitoring platform 300 may restrict access to metrics and insights in the application page for one or more users of the group.

In such cases, the health monitoring platform 300 may prevent the GUI 800 of user device 106 associated with the one or more users from displaying the physiological data during the restriction period. The restriction period may identify a time when the restriction period begins and ends. The restriction period may allow the application pages to be customizable for each user 102 such that the administrator may select what elements of the applications may be displayed to the user device 106. For example, the health monitoring platform 300 may prevent the GUI 800 from displaying messages 810 associated with the Readiness Score or Sleep Score to the user 102 on game-day or other instances where the scores may have a physiological impact on the user's performance. In other examples, the health monitoring platform 300 may prevent the GUI 800 from displaying content to obscure data involved in research. In some cases, the health monitoring platform 300 may enable the GUI 800 to display data upload status, battery life, and other settings, but disable the GUI 800 from displaying metrics and insights. After the restriction period expires, the GUI 800 may display again the metrics and insights including current and past metrics.

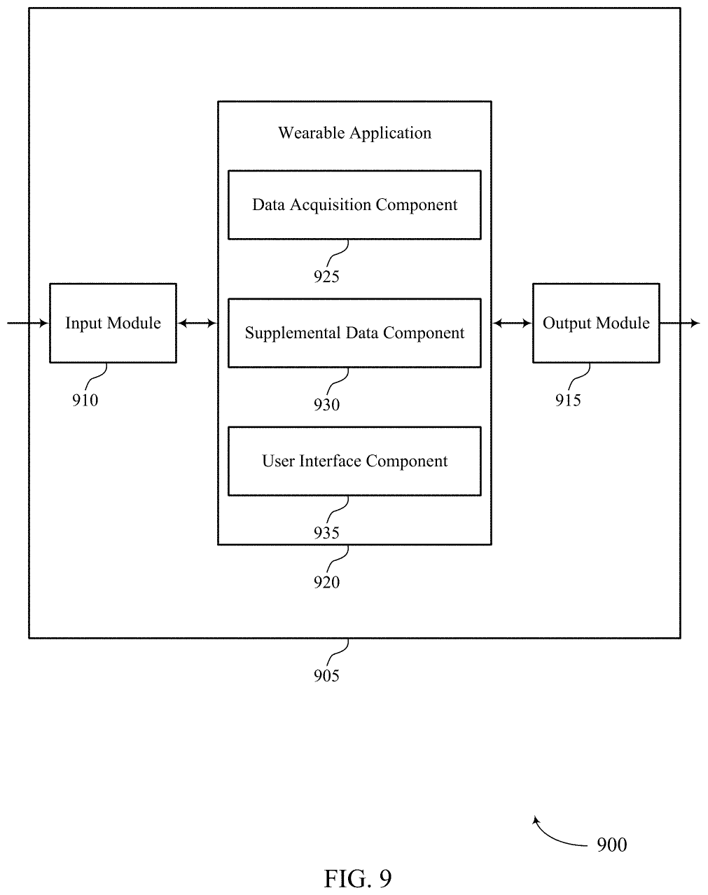

FIG. 9 shows a block diagram 900 of a device 905 that supports a health monitoring platform in accordance with aspects of the present disclosure. The device 905 may include an input module 910, an output module 915, and a wearable application 920. The device 905 may also include a processor. Each of these components may be in communication with one another (e.g., via one or more buses).

The input module 910 may provide a means for receiving information such as packets, user data, control information, or any combination thereof associated with various information channels (e.g., control channels, data channels, information channels related to illness detection techniques). Information may be passed on to other components of the device 905. The input module 910 may utilize a single antenna or a set of multiple antennas.

The output module 915 may provide a means for transmitting signals generated by other components of the device 905. For example, the output module 915 may transmit information such as packets, user data, control information, or any combination thereof associated with various information channels (e.g., control channels, data channels, information channels related to illness detection techniques). In some examples, the output module 915 may be co-located with the input module 910 in a transceiver module. The output module 915 may utilize a single antenna or a set of multiple antennas.

For example, the wearable application 920 may include a data acquisition component 925, a supplemental data component 930, a user interface component 935, or any combination thereof. In some examples, the wearable application 920, or various components thereof, may be configured to perform various operations (e.g., receiving, monitoring, transmitting) using or otherwise in cooperation with the input module 910, the output module 915, or both. For example, the wearable application 920 may receive information from the input module 910, send information to the output module 915, or be integrated in combination with the input module 910, the output module 915, or both to receive information, transmit information, or perform various other operations as described herein.

The data acquisition component 925 may be configured as or otherwise support a means for receiving physiological data associated with one or more users, the physiological data being continuously collected via one or more wearable devices associated with the respective one or more users. The supplemental data component 930 may be configured as or otherwise support a means for receiving, via one or more user devices associated with the one or more users, supplemental data associated with the physiological data for the one or more users, the supplemental data comprising indications of events, subjective attributes, or both. The user interface component 935 may be configured as or otherwise support a means for causing a GUI of an administrator device to display at least a portion of the physiological data. The user interface component 935 may be configured as or otherwise support a means for causing the GUI of the administrator device to display at least a portion of the supplemental data in conjunction with the physiological data, wherein the GUI indicates one or more respective subsets of the physiological data that correspond to one or more respective subsets of the supplemental data.

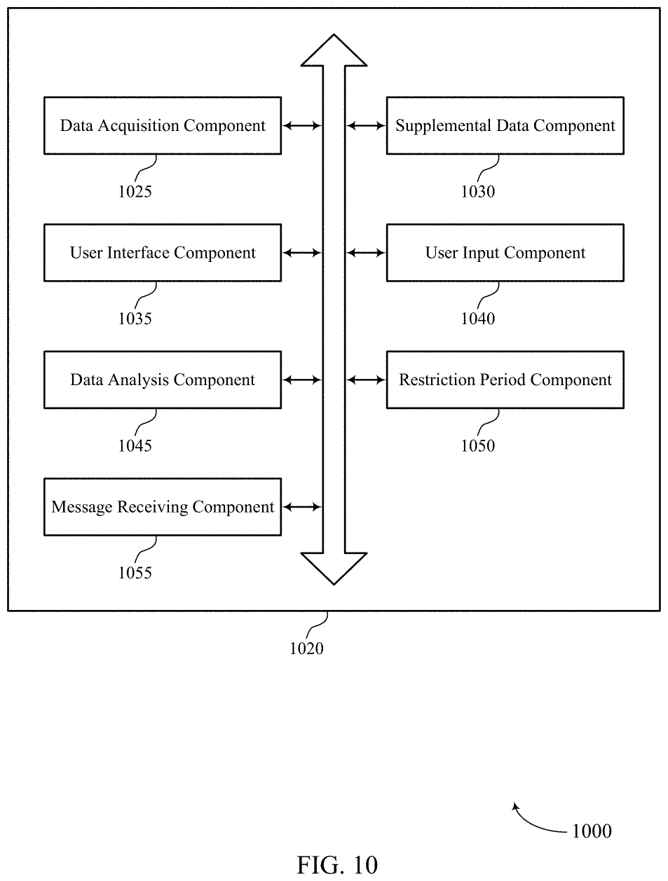

FIG. 10 shows a block diagram 1000 of a wearable application 1020 that supports a health monitoring platform in accordance with aspects of the present disclosure. The wearable application 1020 may be an example of aspects of a wearable application or a wearable application 920, or both, as described herein. The wearable application 1020, or various components thereof, may be an example of means for performing various aspects of health monitoring platform as described herein. For example, the wearable application 1020 may include a data acquisition component 1025, a supplemental data component 1030, a user interface component 1035, a user input component 1040, a data analysis component 1045, a restriction period component 1050, a message receiving component 1055, or any combination thereof. Each of these components may communicate, directly or indirectly, with one another (e.g., via one or more buses).

The data acquisition component 1025 may be configured as or otherwise support a means for receiving physiological data associated with one or more users, the physiological data being continuously collected via one or more wearable devices associated with the respective one or more users. The supplemental data component 1030 may be configured as or otherwise support a means for receiving, via one or more user devices associated with the one or more users, supplemental data associated with the physiological data for the one or more users, the supplemental data comprising indications of events, subjective attributes, or both. The user interface component 1035 may be configured as or otherwise support a means for causing a GUI of an administrator device to display at least a portion of the physiological data. In some examples, the user interface component 1035 may be configured as or otherwise support a means for causing the GUI of the administrator device to display at least a portion of the supplemental data in conjunction with the physiological data, wherein the GUI indicates one or more respective subsets of the physiological data that correspond to one or more respective subsets of the supplemental data.

In some examples, the user input component 1040 may be configured as or otherwise support a means for receiving, via the administrator device, a user input indicating additional supplemental data associated with the physiological data and a time interval associated with the additional supplemental data. In some examples, the user interface component 1035 may be configured as or otherwise support a means for causing the GUI of the administrator device to display the additional supplemental data in conjunction with the time interval associated with the physiological data based at least in part on receiving the user input.

In some examples, the data analysis component 1045 may be configured as or otherwise support a means for determining that the physiological data associated with a user of the one or more users satisfies one or more thresholds. In some examples, the user interface component 1035 may be configured as or otherwise support a means for causing the GUI of the administrator device to display an alert associated with the user based at least in part on determining that the physiological data associated with the user satisfies the one or more thresholds.

In some examples, the user input component 1040 may be configured as or otherwise support a means for receiving, via the administrator device, a user input comprising the one or more thresholds, wherein determining the satisfaction of the one or more thresholds is based at least in part on receiving the user input.

In some examples, the data analysis component 1045 may be configured as or otherwise support a means for determining that the physiological data associated with a user of the one or more users satisfies one or more thresholds. In some examples, the user interface component 1035 may be configured as or otherwise support a means for causing the GUI of a user device associated with the user to display a message based at least in part on determining that the physiological data associated with the user satisfies the one or more thresholds.

In some examples, the user input component 1040 may be configured as or otherwise support a means for receiving, via the administrator device, a user input comprising the one or more thresholds. In some examples, the message receiving component 1055 may be configured as or otherwise support a means for receiving, via the administrator device, one or more messages associated with the one or more thresholds, the one or more messages configured to be communicated upon satisfaction of the respective one or more thresholds, the message included within the one or more messages, wherein causing the GUI of the user device to display the message is based at least in part on receiving the user input and the one or more messages via the administrator device.

In some examples, the message comprises additional information associated with physiological data associated with the user, a link to an external document or survey, medical guidance, or any combination thereof.

In some examples, the restriction period component 1050 may be configured as or otherwise support a means for receiving an indication of a restriction period from the administrator device, the restriction period comprising a time duration that one or more users are unable to view physiological data collected via the wearable device associated with each respective user. In some examples, the user interface component 1035 may be configured as or otherwise support a means for preventing GUIs of user devices associated with the one or more users from displaying the physiological data during the restriction period.

In some examples, the user input component 1040 may be configured as or otherwise support a means for receiving, via the administrator device, an indication of one or more data sets, each data set of the one or more data sets comprising a set of parameters associated with the physiological data, wherein causing the GUI of the administrator device to display at least the portion of the physiological data comprises causing the GUI of the administrator device to display at least the portion of the physiological data corresponding to a data set of the one or more data sets.

In some examples, the supplemental data comprises one or more tags associated with the physiological data.

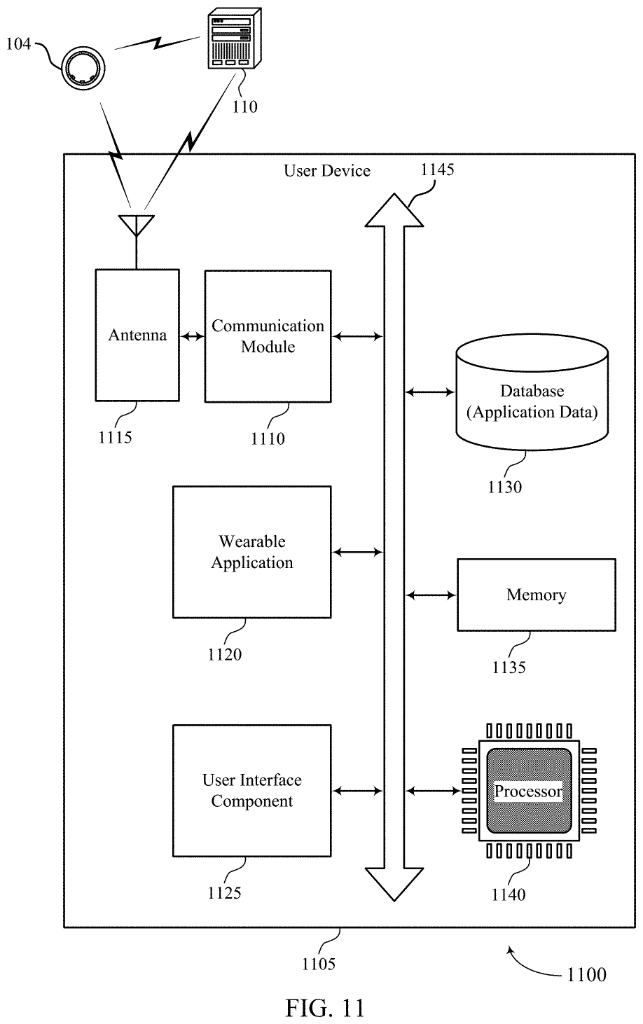

FIG. 11 shows a diagram of a system 1100 including a device 1105 that supports a health monitoring platform in accordance with aspects of the present disclosure. The device 1105 may be an example of or include the components of a device 905 as described herein. The device 1105 may include an example of a user device 106, such as the user device 106 shown and describe in FIGS. 1-3. The device 1105 may include components for bi-directional communications with a ring 104 and a server 110, including components for transmitting and receiving communications, such as a wearable application 1120, a communication module 1110, an antenna 1115, a user interface component 1125, a database (application data) 1130, a memory 1135, and a processor 1140. These components may be in electronic communication or otherwise coupled (e.g., operatively, communicatively, functionally, electronically, electrically) via one or more buses (e.g., a bus 1145).

The communication module 1110 may manage input and output signals for the device 1105 via the antenna 1115. The communication module 1110 may include an example of the communication module 220-b of the user device 106 shown and described in FIG. 2. In this regard, the communication module 1110 may manage communications with the ring 104 and the server 110, as illustrated in FIG. 2. The communication module 1110 may also manage peripherals not integrated into the device 1105. In some cases, the communication module 1110 may represent a physical connection or port to an external peripheral. In some cases, the communication module 1110 may utilize an operating system such as iOS®, ANDROID®, MS-DOS®, MS-WINDOWS®, OS/2®, UNIX®, LINUX®, or another known operating system. In other cases, the communication module 1110 may represent or interact with a wearable device (e.g., ring 104), modem, a keyboard, a mouse, a touchscreen, or a similar device. In some cases, the communication module 1110 may be implemented as part of the processor 1140. In some examples, a user may interact with the device 1105 via the communication module 1110, user interface component 1125, or via hardware components controlled by the communication module 1110.

In some cases, the device 1105 may include a single antenna 1115. However, in some other cases, the device 1105 may have more than one antenna 1115, that may be capable of concurrently transmitting or receiving multiple wireless transmissions. The communication module 1110 may communicate bi-directionally, via the one or more antennas 1115, wired, or wireless links as described herein. For example, the communication module 1110 may represent a wireless transceiver and may communicate bi-directionally with another wireless transceiver. The communication module 1110 may also include a modem to modulate the packets, to provide the modulated packets to one or more antennas 1115 for transmission, and to demodulate packets received from the one or more antennas 1115.

The user interface component 1125 may manage data storage and processing in a database 1130. In some cases, a user may interact with the user interface component 1125. In other cases, the user interface component 1125 may operate automatically without user interaction. The database 1130 may be an example of a single database, a distributed database, multiple distributed databases, a data store, a data lake, or an emergency backup database.

The memory 1135 may include RAM and ROM. The memory 1135 may store computer-readable, computer-executable software including instructions that, when executed, cause the processor 1140 to perform various functions described herein. In some cases, the memory 1135 may contain, among other things, a BIOS that may control basic hardware or software operation such as the interaction with peripheral components or devices.

The processor 1140 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 1140 may be configured to operate a memory array using a memory controller. In other cases, a memory controller may be integrated into the processor 1140. The processor 1140 may be configured to execute computer-readable instructions stored in a memory 1135 to perform various functions (e.g., functions or tasks supporting a method and system for sleep staging algorithms).

For example, the wearable application 1120 may be configured as or otherwise support a means for receiving physiological data associated with one or more users, the physiological data being continuously collected via one or more wearable devices associated with the respective one or more users. The wearable application 1120 may be configured as or otherwise support a means for receiving, via one or more user devices associated with the one or more users, supplemental data associated with the physiological data for the one or more users, the supplemental data comprising indications of events, subjective attributes, or both. The wearable application 1120 may be configured as or otherwise support a means for causing a GUI of an administrator device to display at least a portion of the physiological data. The wearable application 1120 may be configured as or otherwise support a means for causing the GUI of the administrator device to display at least a portion of the supplemental data in conjunction with the physiological data, wherein the GUI indicates one or more respective subsets of the physiological data that correspond to one or more respective subsets of the supplemental data.

By including or configuring the wearable application 1120 in accordance with examples as described herein, the device 1105 may support techniques for improved illness detection/prediction. In particular, techniques described herein may enable a health monitoring platform that is configured to alert users and/or administrators about the physiological data of the user. As such, techniques described herein may reduce a spread of illness, reduce a severity of illness, optimize user performance, increase the health and wellness of the user, and the like.

The wearable application 1120 may include an application (e.g., “app”), program, software, or other component that is configured to facilitate communications with a ring 104, server 110, other user devices 106, and the like. For example, the wearable application 1120 may include an application executable on a user device 106 that is configured to receive data (e.g., physiological data) from a ring 104, perform processing operations on the received data, transmit and receive data with the servers 110, and cause presentation of data to a user 102.

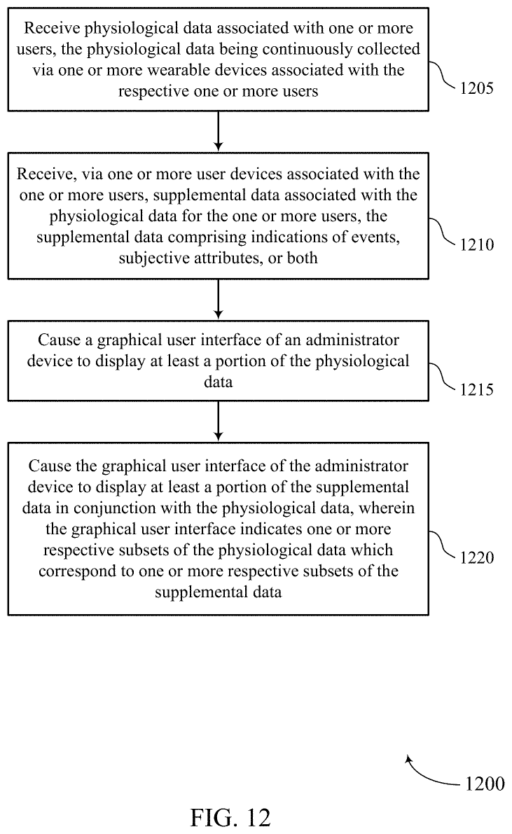

FIG. 12 shows a flowchart illustrating a method 1200 that supports a health monitoring platform in accordance with aspects of the present disclosure. The operations of the method 1200 may be implemented by a user device or its components as described herein. For example, the operations of the method 1200 may be performed by a user device as described with reference to FIGS. 1 through 11. In some examples, a user device may execute a set of instructions to control the functional elements of the user device to perform the described functions. Additionally, or alternatively, the user device may perform aspects of the described functions using special-purpose hardware.

At 1205, the method may include receiving physiological data associated with one or more users, the physiological data being continuously collected via one or more wearable devices associated with the respective one or more users. The operations of 1205 may be performed in accordance with examples as disclosed herein. In some examples, aspects of the operations of 1205 may be performed by a data acquisition component 1025 as described with reference to FIG. 10.

At 1210, the method may include receiving, via one or more user devices associated with the one or more users, supplemental data associated with the physiological data for the one or more users, the supplemental data comprising indications of events, subjective attributes, or both. The operations of 1210 may be performed in accordance with examples as disclosed herein. In some examples, aspects of the operations of 1210 may be performed by a supplemental data component 1030 as described with reference to FIG. 10.

At 1215, the method may include causing a GUI of an administrator device to display at least a portion of the physiological data. The operations of 1215 may be performed in accordance with examples as disclosed herein. In some examples, aspects of the operations of 1215 may be performed by a user interface component 1035 as described with reference to FIG. 10.

At 1220, the method may include causing the GUI of the administrator device to display at least a portion of the supplemental data in conjunction with the physiological data, wherein the GUI indicates one or more respective subsets of the physiological data that correspond to one or more respective subsets of the supplemental data. The operations of 1220 may be performed in accordance with examples as disclosed herein. In some examples, aspects of the operations of 1220 may be performed by a user interface component 1035 as described with reference to FIG. 10.

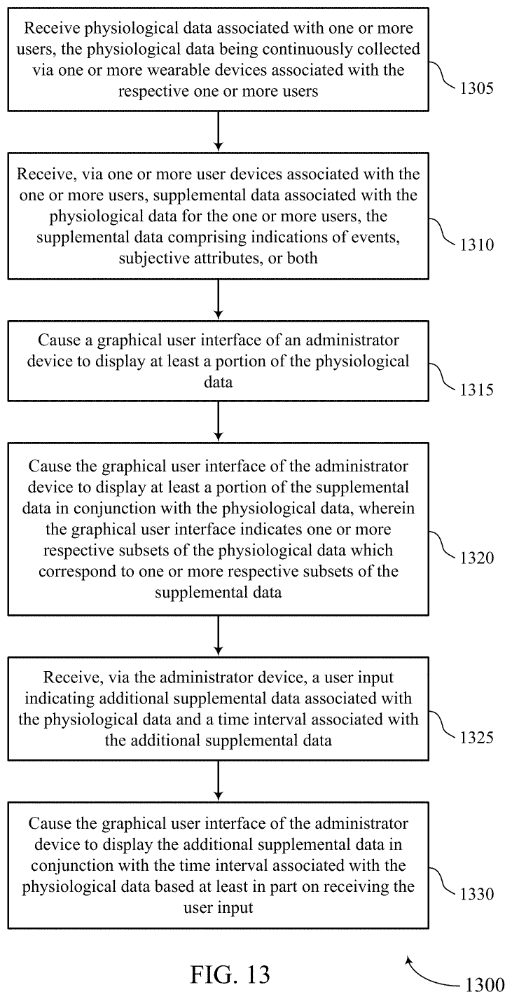

FIG. 13 shows a flowchart illustrating a method 1300 that supports a health monitoring platform in accordance with aspects of the present disclosure. The operations of the method 1300 may be implemented by a user device or its components as described herein. For example, the operations of the method 1300 may be performed by a user device as described with reference to FIGS. 1 through 11. In some examples, a user device may execute a set of instructions to control the functional elements of the user device to perform the described functions. Additionally, or alternatively, the user device may perform aspects of the described functions using special-purpose hardware.

At 1305, the method may include receiving physiological data associated with one or more users, the physiological data being continuously collected via one or more wearable devices associated with the respective one or more users. The operations of 1305 may be performed in accordance with examples as disclosed herein. In some examples, aspects of the operations of 1305 may be performed by a data acquisition component 1025 as described with reference to FIG. 10.

At 1310, the method may include receiving, via one or more user devices associated with the one or more users, supplemental data associated with the physiological data for the one or more users, the supplemental data comprising indications of events, subjective attributes, or both. The operations of 1310 may be performed in accordance with examples as disclosed herein. In some examples, aspects of the operations of 1310 may be performed by a supplemental data component 1030 as described with reference to FIG. 10.

At 1315, the method may include causing a GUI of an administrator device to display at least a portion of the physiological data. The operations of 1315 may be performed in accordance with examples as disclosed herein. In some examples, aspects of the operations of 1315 may be performed by a user interface component 1035 as described with reference to FIG. 10.

At 1320, the method may include causing the GUI of the administrator device to display at least a portion of the supplemental data in conjunction with the physiological data, wherein the GUI indicates one or more respective subsets of the physiological data that correspond to one or more respective subsets of the supplemental data. The operations of 1320 may be performed in accordance with examples as disclosed herein. In some examples, aspects of the operations of 1320 may be performed by a user interface component 1035 as described with reference to FIG. 10.

At 1325, the method may include receiving, via the administrator device, a user input indicating additional supplemental data associated with the physiological data and a time interval associated with the additional supplemental data. The operations of 1325 may be performed in accordance with examples as disclosed herein. In some examples, aspects of the operations of 1325 may be performed by a user input component 1040 as described with reference to FIG. 10.

At 1330, the method may include causing the GUI of the administrator device to display the additional supplemental data in conjunction with the time interval associated with the physiological data based at least in part on receiving the user input. The operations of 1330 may be performed in accordance with examples as disclosed herein. In some examples, aspects of the operations of 1330 may be performed by a user interface component 1035 as described with reference to FIG. 10.

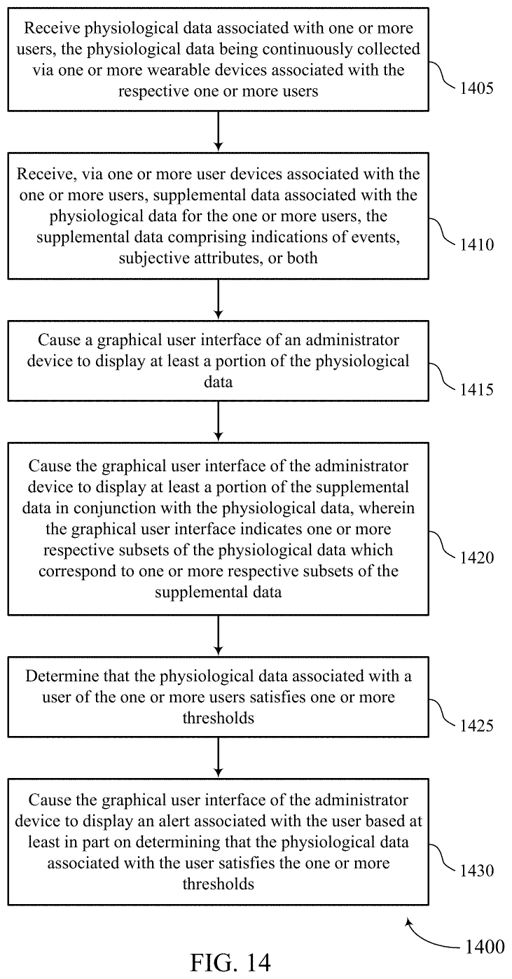

FIG. 14 shows a flowchart illustrating a method 1400 that supports a health monitoring platform in accordance with aspects of the present disclosure. The operations of the method 1400 may be implemented by a user device or its components as described herein. For example, the operations of the method 1400 may be performed by a user device as described with reference to FIGS. 1 through 11. In some examples, a user device may execute a set of instructions to control the functional elements of the user device to perform the described functions. Additionally, or alternatively, the user device may perform aspects of the described functions using special-purpose hardware.

At 1405, the method may include receiving physiological data associated with one or more users, the physiological data being continuously collected via one or more wearable devices associated with the respective one or more users. The operations of 1405 may be performed in accordance with examples as disclosed herein. In some examples, aspects of the operations of 1405 may be performed by a data acquisition component 1025 as described with reference to FIG. 10.

At 1410, the method may include receiving, via one or more user devices associated with the one or more users, supplemental data associated with the physiological data for the one or more users, the supplemental data comprising indications of events, subjective attributes, or both. The operations of 1410 may be performed in accordance with examples as disclosed herein. In some examples, aspects of the operations of 1410 may be performed by a supplemental data component 1030 as described with reference to FIG. 10.

At 1415, the method may include causing a GUI of an administrator device to display at least a portion of the physiological data. The operations of 1415 may be performed in accordance with examples as disclosed herein. In some examples, aspects of the operations of 1415 may be performed by a user interface component 1035 as described with reference to FIG. 10.

At 1420, the method may include causing the GUI of the administrator device to display at least a portion of the supplemental data in conjunction with the physiological data, wherein the GUI indicates one or more respective subsets of the physiological data that correspond to one or more respective subsets of the supplemental data. The operations of 1420 may be performed in accordance with examples as disclosed herein. In some examples, aspects of the operations of 1420 may be performed by a user interface component 1035 as described with reference to FIG. 10.

At 1425, the method may include determining that the physiological data associated with a user of the one or more users satisfies one or more thresholds. The operations of 1425 may be performed in accordance with examples as disclosed herein. In some examples, aspects of the operations of 1425 may be performed by a data analysis component 1045 as described with reference to FIG. 10.

At 1430, the method may include causing the GUI of the administrator device to display an alert associated with the user based at least in part on determining that the physiological data associated with the user satisfies the one or more thresholds. The operations of 1430 may be performed in accordance with examples as disclosed herein. In some examples, aspects of the operations of 1430 may be performed by a user interface component 1035 as described with reference to FIG. 10.

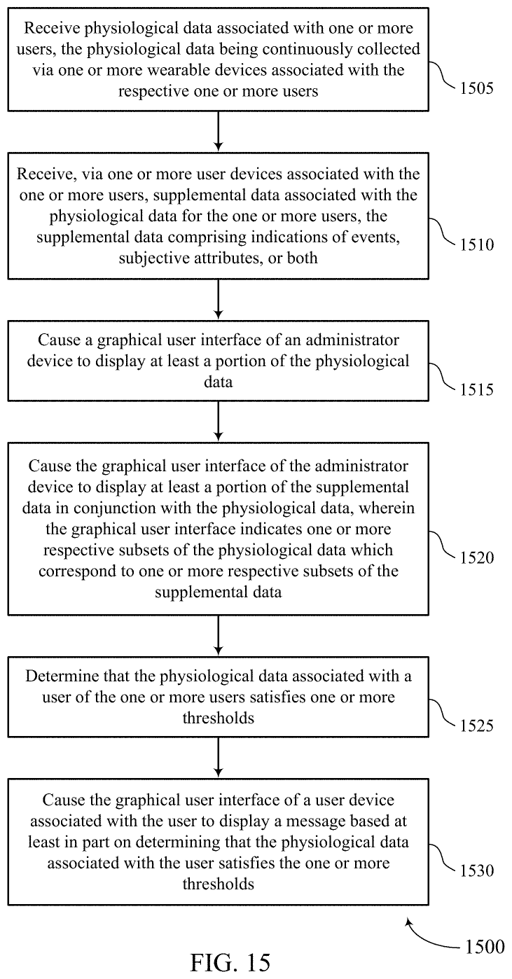

FIG. 15 shows a flowchart illustrating a method 1500 that supports a health monitoring platform in accordance with aspects of the present disclosure. The operations of the method 1500 may be implemented by a user device or its components as described herein. For example, the operations of the method 1500 may be performed by a user device as described with reference to FIGS. 1 through 11. In some examples, a user device may execute a set of instructions to control the functional elements of the user device to perform the described functions. Additionally, or alternatively, the user device may perform aspects of the described functions using special-purpose hardware.

At 1505, the method may include receiving physiological data associated with one or more users, the physiological data being continuously collected via one or more wearable devices associated with the respective one or more users. The operations of 1505 may be performed in accordance with examples as disclosed herein. In some examples, aspects of the operations of 1505 may be performed by a data acquisition component 1025 as described with reference to FIG. 10.

At 1510, the method may include receiving, via one or more user devices associated with the one or more users, supplemental data associated with the physiological data for the one or more users, the supplemental data comprising indications of events, subjective attributes, or both. The operations of 1510 may be performed in accordance with examples as disclosed herein. In some examples, aspects of the operations of 1510 may be performed by a supplemental data component 1030 as described with reference to FIG. 10.

At 1515, the method may include causing a GUI of an administrator device to display at least a portion of the physiological data. The operations of 1515 may be performed in accordance with examples as disclosed herein. In some examples, aspects of the operations of 1515 may be performed by a user interface component 1035 as described with reference to FIG. 10.

At 1520, the method may include causing the GUI of the administrator device to display at least a portion of the supplemental data in conjunction with the physiological data, wherein the GUI indicates one or more respective subsets of the physiological data that correspond to one or more respective subsets of the supplemental data. The operations of 1520 may be performed in accordance with examples as disclosed herein. In some examples, aspects of the operations of 1520 may be performed by a user interface component 1035 as described with reference to FIG. 10.

At 1525, the method may include determining that the physiological data associated with a user of the one or more users satisfies one or more thresholds. The operations of 1525 may be performed in accordance with examples as disclosed herein. In some examples, aspects of the operations of 1525 may be performed by a data analysis component 1045 as described with reference to FIG. 10.

At 1530, the method may include causing the GUI of a user device associated with the user to display a message based at least in part on determining that the physiological data associated with the user satisfies the one or more thresholds. The operations of 1530 may be performed in accordance with examples as disclosed herein. In some examples, aspects of the operations of 1530 may be performed by a user interface component 1035 as described with reference to FIG. 10.

A method is described. The method may include receiving physiological data associated with one or more users, the physiological data being continuously collected via one or more wearable devices associated with the respective one or more users, receiving, via one or more user devices associated with the one or more users, supplemental data associated with the physiological data for the one or more users, the supplemental data comprising indications of events, subjective attributes, or both, causing a GUI of an administrator device to display at least a portion of the physiological data, and causing the GUI of the administrator device to display at least a portion of the supplemental data in conjunction with the physiological data, wherein the GUI indicates one or more respective subsets of the physiological data that correspond to one or more respective subsets of the supplemental data.

An apparatus is described. The apparatus may include a processor, memory coupled with the processor, and instructions stored in the memory. The instructions may be executable by the processor to cause the apparatus to receive physiological data associated with one or more users, the physiological data being continuously collected via one or more wearable devices associated with the respective one or more users, receive, via one or more user devices associated with the one or more users, supplemental data associated with the physiological data for the one or more users, the supplemental data comprising indications of events, subjective attributes, or both, cause a GUI of an administrator device to display at least a portion of the physiological data, and cause the GUI of the administrator device to display at least a portion of the supplemental data in conjunction with the physiological data, wherein the GUI indicates one or more respective subsets of the physiological data that correspond to one or more respective subsets of the supplemental data.

Another apparatus is described. The apparatus may include means for receiving physiological data associated with one or more users, the physiological data being continuously collected via one or more wearable devices associated with the respective one or more users, means for receiving, via one or more user devices associated with the one or more users, supplemental data associated with the physiological data for the one or more users, the supplemental data comprising indications of events, subjective attributes, or both, means for causing a GUI of an administrator device to display at least a portion of the physiological data, and means for causing the GUI of the administrator device to display at least a portion of the supplemental data in conjunction with the physiological data, wherein the GUI indicates one or more respective subsets of the physiological data that correspond to one or more respective subsets of the supplemental data.

A non-transitory computer-readable medium storing code is described. The code may include instructions executable by a processor to receive physiological data associated with one or more users, the physiological data being continuously collected via one or more wearable devices associated with the respective one or more users, receive, via one or more user devices associated with the one or more users, supplemental data associated with the physiological data for the one or more users, the supplemental data comprising indications of events, subjective attributes, or both, cause a GUI of an administrator device to display at least a portion of the physiological data, and cause the GUI of the administrator device to display at least a portion of the supplemental data in conjunction with the physiological data, wherein the GUI indicates one or more respective subsets of the physiological data that correspond to one or more respective subsets of the supplemental data.

Some examples of the method, apparatuses, and non-transitory computer-readable medium described herein may further include operations, features, means, or instructions for receiving, via the administrator device, a user input indicating additional supplemental data associated with the physiological data and a time interval associated with the additional supplemental data and causing the GUI of the administrator device to display the additional supplemental data in conjunction with the time interval associated with the physiological data based at least in part on receiving the user input.

Some examples of the method, apparatuses, and non-transitory computer-readable medium described herein may further include operations, features, means, or instructions for determining that the physiological data associated with a user of the one or more users satisfies one or more thresholds and causing the GUI of the administrator device to display an alert associated with the user based at least in part on determining that the physiological data associated with the user satisfies the one or more thresholds.

Some examples of the method, apparatuses, and non-transitory computer-readable medium described herein may further include operations, features, means, or instructions for receiving, via the administrator device, a user input comprising the one or more thresholds, wherein determining the satisfaction of the one or more thresholds may be based at least in part on receiving the user input.

Some examples of the method, apparatuses, and non-transitory computer-readable medium described herein may further include operations, features, means, or instructions for determining that the physiological data associated with a user of the one or more users satisfies one or more thresholds and causing the GUI of a user device associated with the user to display a message based at least in part on determining that the physiological data associated with the user satisfies the one or more thresholds.

Some examples of the method, apparatuses, and non-transitory computer-readable medium described herein may further include operations, features, means, or instructions for receiving, via the administrator device, a user input comprising the one or more thresholds and receiving, via the administrator device, one or more messages associated with the one or more thresholds, the one or more messages configured to be communicated upon satisfaction of the respective one or more thresholds, the message included within the one or more messages, wherein causing the GUI of the user device to display the message may be based at least in part on receiving the user input and the one or more messages via the administrator device.

In some examples of the method, apparatuses, and non-transitory computer-readable medium described herein, the message comprises additional information associated with physiological data associated with the user, a link to an external document or survey, medical guidance, or any combination thereof.

Some examples of the method, apparatuses, and non-transitory computer-readable medium described herein may further include operations, features, means, or instructions for receiving an indication of a restriction period from the administrator device, the restriction period comprising a time duration that one or more users may be unable to view physiological data collected via the wearable device associated with each respective user and preventing GUIs of user devices associated with the one or more users from displaying the physiological data during the restriction period.

Some examples of the method, apparatuses, and non-transitory computer-readable medium described herein may further include operations, features, means, or instructions for receiving, via the administrator device, an indication of one or more data sets, each data set of the one or more data sets comprising a set of parameters associated with the physiological data, wherein causing the GUI of the administrator device to display at least the portion of the physiological data comprises causing the GUI of the administrator device to display at least the portion of the physiological data corresponding to a data set of the one or more data sets.

In some examples of the method, apparatuses, and non-transitory computer-readable medium described herein, the supplemental data comprises one or more tags associated with the physiological data.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method, comprising:
receiving physiological data associated with one or more users, the physiological data being continuously collected via one or more wearable devices associated with the respective one or more users;
receiving, via one or more user devices associated with the one or more users, supplemental data associated with the physiological data for the one or more users, the supplemental data comprising indications of events, subjective attributes, or both;
causing a graphical user interface of an administrator device to display at least a portion of the physiological data; and
causing the graphical user interface of the administrator device to display at least a portion of the supplemental data in conjunction with the physiological data, wherein the graphical user interface indicates one or more respective subsets of the physiological data that correspond to one or more respective subsets of the supplemental data.

2. The method of claim 1, further comprising:
receiving, via the administrator device, a user input indicating additional supplemental data associated with the physiological data and a time interval associated with the additional supplemental data; and
causing the graphical user interface of the administrator device to display the additional supplemental data in conjunction with the time interval associated with the physiological data based at least in part on receiving the user input.

3. The method of claim 1, further comprising:
determining that the physiological data associated with a user of the one or more users satisfies one or more thresholds; and
causing the graphical user interface of the administrator device to display an alert associated with the user based at least in part on determining that the physiological data associated with the user satisfies the one or more thresholds.

4. The method of claim 3, further comprising:
receiving, via the administrator device, a user input comprising the one or more thresholds, wherein determining the satisfaction of the one or more thresholds is based at least in part on receiving the user input.

5. The method of claim 1, further comprising:
determining that the physiological data associated with a user of the one or more users satisfies one or more thresholds; and
causing the graphical user interface of a user device associated with the user to display a message based at least in part on determining that the physiological data associated with the user satisfies the one or more thresholds.

6. The method of claim 5, further comprising:
receiving, via the administrator device, a user input comprising the one or more thresholds; and
receiving, via the administrator device, one or more messages associated with the one or more thresholds, the one or more messages configured to be communicated upon satisfaction of the respective one or more thresholds, the message included within the one or more messages, wherein causing the graphical user interface of the user device to display the message is based at least in part on receiving the user input and the one or more messages via the administrator device.

7. The method of claim 5, wherein the message comprises additional information associated with physiological data associated with the user, a link to an external document or survey, medical guidance, or any combination thereof.

8. The method of claim 1, further comprising:
receiving an indication of a restriction period from the administrator device, the restriction period comprising a time duration that one or more users are unable to view physiological data collected via the wearable device associated with each respective user; and
preventing graphical user interfaces of user devices associated with the one or more users from displaying the physiological data during the restriction period.

9. The method of claim 1, further comprising:
receiving, via the administrator device, an indication of one or more data sets, each data set of the one or more data sets comprising a set of parameters associated with the physiological data, wherein causing the graphical user interface of the administrator device to display at least the portion of the physiological data comprises causing the graphical user interface of the administrator device to display at least the portion of the physiological data corresponding to a data set of the one or more data sets.

10. The method of claim 1, wherein the supplemental data comprises one or more tags associated with the physiological data.

11. An apparatus, comprising:
a processor;
memory coupled with the processor; and
instructions stored in the memory and executable by the processor to cause the apparatus to:
receive physiological data associated with one or more users, the physiological data being continuously collected via one or more wearable devices associated with the respective one or more users;
receive, via one or more user devices associated with the one or more users, supplemental data associated with the physiological data for the one or more users, the supplemental data comprising indications of events, subjective attributes, or both;
cause a graphical user interface of an administrator device to display at least a portion of the physiological data; and
cause the graphical user interface of the administrator device to display at least a portion of the supplemental data in conjunction with the physiological data, wherein the graphical user interface indicates one or more respective subsets of the physiological data that correspond to one or more respective subsets of the supplemental data.

12. The apparatus of claim 11, wherein the instructions are further executable by the processor to cause the apparatus to:
receive, via the administrator device, a user input indicating additional supplemental data associated with the physiological data and a time interval associated with the additional supplemental data; and
cause the graphical user interface of the administrator device to display the additional supplemental data in conjunction with the time interval associated with the physiological data based at least in part on receiving the user input.

13. The apparatus of claim 11, wherein the instructions are further executable by the processor to cause the apparatus to:
determine that the physiological data associated with a user of the one or more users satisfies one or more thresholds; and
cause the graphical user interface of the administrator device to display an alert associated with the user based at least in part on determining that the physiological data associated with the user satisfies the one or more thresholds.

14. The apparatus of claim 13, wherein the instructions are further executable by the processor to cause the apparatus to:
receive, via the administrator device, a user input comprising the one or more thresholds, wherein determining the satisfaction of the one or more thresholds is based at least in part on receiving the user input.

15. The apparatus of claim 11, wherein the instructions are further executable by the processor to cause the apparatus to:
determine that the physiological data associated with a user of the one or more users satisfies one or more thresholds; and
cause the graphical user interface of a user device associated with the user to display a message based at least in part on determining that the physiological data associated with the user satisfies the one or more thresholds.

16. The apparatus of claim 15, wherein the instructions are further executable by the processor to cause the apparatus to:
receive, via the administrator device, a user input comprising the one or more thresholds; and
receive, via the administrator device, one or more messages associated with the one or more thresholds, the one or more messages configured to be communicated upon satisfaction of the respective one or more thresholds, the message included within the one or more messages, wherein causing the graphical user interface of the user device to display the message is based at least in part on receiving the user input and the one or more messages via the administrator device.

17. The apparatus of claim 15, wherein the message comprises additional information associated with physiological data associated with the user, a link to an external document or survey, medical guidance, or any combination thereof.

18. The apparatus of claim 11, wherein the instructions are further executable by the processor to cause the apparatus to:
receive an indication of a restriction period from the administrator device, the restriction period comprising a time duration that one or more users are unable to view physiological data collected via the wearable device associated with each respective user; and
prevent graphical user interfaces of user devices associated with the one or more users from displaying the physiological data during the restriction period.

19. The apparatus of claim 11, wherein the instructions are further executable by the processor to cause the apparatus to:
receive, via the administrator device, an indication of one or more data sets, each data set of the one or more data sets comprising a set of parameters associated with the physiological data, wherein causing the graphical user interface of the administrator device to display at least the portion of the physiological data comprises causing the graphical user interface of the administrator device to display at least the portion of the physiological data corresponding to a data set of the one or more data sets.

20. A non-transitory computer-readable medium storing code, the code comprising instructions executable by a processor to:
receive physiological data associated with one or more users, the physiological data being continuously collected via one or more wearable devices associated with the respective one or more users;
receive, via one or more user devices associated with the one or more users, supplemental data associated with the physiological data for the one or more users, the supplemental data comprising indications of events, subjective attributes, or both;
cause a graphical user interface of an administrator device to display at least a portion of the physiological data; and
cause the graphical user interface of the administrator device to display at least a portion of the supplemental data in conjunction with the physiological data, wherein the graphical user interface indicates one or more respective subsets of the physiological data that correspond to one or more respective subsets of the supplemental data.
